(19) United States
(12) Patent Application Publication
(10) Pub. No.: US 2024/0075311 A1
(43) Pub. Date: Mar. 7, 2024

(54) ELECTROENCEPHALOGRAM (EEG) BASED TRANSCRANIAL MAGNETIC STIMULATION (TMS) DEVICES

(71) Applicant: WAVE NEUROSCIENCE, INC., Newport Beach, CA (US)

(72) Inventors: James William PHILLIPS, Fountain Valley, CA (US); Alexander RING, Newport Beach, CA (US)

(73) Assignee: WAVE NEUROSCIENCE, INC., Newport Beach, CA (US)

(21) Appl. No.: 18/280,922

(22) PCT Filed: Mar. 25, 2022
(86) PCT No.: PCT/US22/71355

§ 371 Date: Sep. 7, 2023

Related U.S. Application Data
(60) Provisional application No. 63/166,944, filed on Mar. 26, 2021.

(51) Int. Cl. A61N 2/12; A61N 2/00

(52) U.S. Cl. CPC A61N 2/12; A61N 2/002; A61N 2/006

(57) ABSTRACT

A system and method for providing transcranial magnetic stimulation (TMS) based on electroencephalogram (EEG) data. The system and method may include a portable neuro-electroencephalogram synchronization therapy (NEST) system for capturing EEG data from a patient. The system and method may also include an electrophysiology database and customized TMS treatment system for receiving the EEG data from the portable NEST system. The electrophysiology database and customized TMS treatment system may determine a TMS treatment based on the received EEG data. The portable NEST system may provide synchronized TMS based on the determined TMS treatment.

### CROSS-REFERENCE

This application claims the benefit of U.S. Provisional Application No. 63/166,944, filed Mar. 26, 2021, entitled METHODS, SYSTEMS, KITS AND APPARATUSES FOR PROVIDING TRANSCRANIAL MAGNETIC STIMULATION (TMS) BASED ON ELECTROENCEPHALOGRAM (EEG) DATA, which application is incorporated herein in its entirety by reference.

This application is related to the following co-pending patent applications: application Serial No. PCT/US22/71357 filed Mar. 25, 2022; and application Serial No. PCT/US22/71358 filed Mar. 25, 2022, which are incorporated herein in their entirely by reference.

### BACKGROUND

### Field

The disclosure relates to devices for providing transcranial magnetic stimulation (TMS) therapy based on electroencephalogram (EEG) data.

### Background

Mental disorders cause serious problems for affected people, their families, and society. Currently, psychiatrists and neurophysiologists treat these disorders with a variety of medications, many of which have significant negative side effects. Mental disorders can be painful, debilitating, and very costly for the affected individual and their family. Approximately one in five adults in the US experiences a mental disorder in a given year. 18.1% of adults in the US experience an anxiety disorder, such as posttraumatic stress disorder, obsessive-compulsive disorder (OCD) and specific phobias. 6.9% of adults in the US have at least one major depressive episode each year. 1.1% of adults in the US live with schizophrenia. In fact, mental disorders are the third most common cause of hospitalization in the US for both youth and adults aged 18-44 and the consequences of lack of treatment are significant. Sadly, suicide is the 10th leading cause of death in the U.S. and the 2nd leading cause of death for those aged 15-24. Each day, approximately 18-22 veterans die by suicide.

A key factor in treatment of mental disorders is proper diagnosis. The standard method of diagnosing mental disorders has been with either the Diagnostic and Statistical Manual of Mental Disorders (DSM) or the International Statistical Classification of Diseases and Related Health Problems (ICD), Chapter 5: Mental and behavioral disorders. Both standards primarily involve diagnosis using conversation with the patient regarding symptoms and behavior. This standard has the disadvantage of being subjectively based on the interviewer's perception, which lessens the diagnostic reliability, sometimes resulting in two clinicians arriving at different diagnoses for the same patient. Additionally, a patient's responses to questions may vary based upon their present situation or life circumstance. Because the DSM and ICD are primarily concerned with the signs and symptoms of mental disorders, rather than the underlying causes, there is a general lack of pathophysiological understanding of mental disorders.

It is apparent that a repeatable and reliable system for the diagnosis of mental disorders that is based on measurable data, independent of the biases and interpretation of an interviewer or a fluctuating situational condition of the patient, would provide significant benefit to patients and to the psychiatric community.

Treatment of these disorders with magnetic fields, such as those generated by transcranial magnetic stimulation (TMS), may generate positive therapeutic responses.

TMS is a generally known non-invasive procedure that uses magnetic pulses to stimulate nerve cells and neuronal circuitry in the brain to improve mental disorders such as depression. Magnetic pulses delivered at regular intervals, described as repetitive magnetic pulses, are referred to as rTMS. Traditionally, coils (e.g., a large electric coil) have been used to generate rTMS. The large electric coil (e.g., electromagnetic coil) may be placed against a patient's scalp when applying stimulation. Some studies have shown that rTMS can reduce the negative symptoms of schizophrenia, obsessive compulsive disorder (OCD), and depression under certain circumstances.

Magnetic fields, especially time and magnitude-varying magnetic fields, can also be generated by movement of one or more magnets. For example, some devices provide TMS by using rotating permanent magnets where magnets are positioned around a patient's head to provide TMS towards regions of the patient's brain to stimulate nerve cells in those specific regions of the brain. In some of these examples, rTMS uses an electromagnet placed on the scalp that generates a series of magnetic field pulses roughly the strength of an MRI scan.

There are also normative electroencephalogram (EEG) databases that have been in existence for several years at a few universities. The normative EEG databases are typically accompanied by clinical scores or evaluations of some kind for EEG data that was collected from patients. These normative EEG databases have been used to aid medical professionals in evaluating neurological statuses of patients prior to and after therapy to adjust therapy as needed based on EEG biofeedback. This deeper knowledge can benefit patients, medical professionals (e.g., clinicians), and the greater neurological or neurotherapy field. These databases are usually based on a wide age range from children up to adults. Examples of universities having these normative EEG databases include New York University (NYU) and the University of Maryland.

What is needed are devices that customize application of TMS based on normative EEG data.

### SUMMARY

The disclosure relates generally to devices for providing transcranial magnetic stimulation (TMS) based on normative electrophysiological measurement such as electroencephalogram (EEG) data. A normative database provides operational feedback to the devices that accounts for deviations from an expected normal parameters in a population.

The device includes: a portable neuro-electroencephalogram synchronization therapy (NEST) system for capturing the EEG data from a patient in communication with a normative EEG database which is operable to customize the application of TMS based on the EEG data compared to the normative EEG data from the database. The electrophysiology database is used to customize TMS treatment delivered from the devices based on the received EEG data. The portable NEST devices may provide synchronized transcranial magnetic stimulation based on the determined TMS treatment.

The portable NEST devices may also provide the synchronized transcranial magnetic stimulation relating to a defined treatment protocol.

Additionally, the portable NEST devices may have a mobile design developed through miniaturization and compacting of components within the portable NEST devices allowing for use of the portable NEST devices at various locations and when the patient may be in-transit between locations. Additionally, the portable NEST devices may be configured as a wearable headgear that provides the TMS treatment during mobility of the patient. A portable power source can also be provided. Safety features can also be provided to prevent misuse of the device.

The portable NEST system may include a magnetic stimulation system having rotating magnets that may generate an alternating magnetic field that provides the synchronized transcranial magnetic stimulation based on the determined TMS treatment.

The NEST systems may also provide the recommended synchronized transcranial magnetic stimulation to the patient. The systems may include an electrophysiology database and customized TMS treatment system for accessing the EEG data for a patient. The electrophysiology database and customized TMS treatment system may also provide bursting analysis of the EEG data, and the electrophysiology database and customized TMS treatment system may determine a TMS treatment based on the bursting analysis and the EEG data. The electrophysiology database and customized TMS treatment system may recommend synchronized transcranial magnetic stimulation based on the determined TMS treatment. Additionally, the electrophysiology database and customized TMS treatment system may use the bursting analysis for diagnosing the patient. In some embodiments, the electrophysiology database and customized TMS treatment system may use the bursting analysis for determining treatment parameters.

The devices can communication with secondary systems, such as an electrophysiology database and customized TMS treatment system for receiving the EEG data from the portable NEST system, and the electrophysiology database and customized TMS treatment system may determine the TMS treatment based on the received EEG data.

A kit for providing TMS based on EEG data is disclosed. The kit may include a portable NEST system configured within a bounding box having a maximum volume with maximum dimensions of about X by about Y by about Z for providing one or more portability features for a patient. The portable NEST system may capture EEG data from the patient, and the portable NEST system may determine a TMS treatment recommendation based on the EEG data or may receive the TMS treatment recommendation from a second system based on the EEG data. The portable NEST system may provide synchronized transcranial magnetic stimulation based on the determined TMS treatment or the recommended TMS treatment. Portability features may include at least one of compact, transportable, movable, mobile, easy to carry, or relatively lightweight. In some embodiments, the portability features may include at least one of positionable and removable, or modular.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

U.S. Pat. No. 9,962,555 issued May 8, 2018 to Charles et al.; and

U.S. Pat. No. 10,835,754 issued Nov. 17, 2020 to Charles et al.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a diagrammatic environment view that depicts an exemplary portable NEST system and an electrophysiology database and customized TMS treatment system communicating via a network, and communicating with various other systems, devices, processes, and information sources according to embodiments of the disclosure;

FIG. 2A is a diagrammatic view that depicts examples of the portable NEST system of FIG. 1 according to embodiments of the disclosure;

FIG. 2B is a diagrammatic environment view that depicts an exemplary portable NEST system and electrophysiology database and customized TMS treatment system communicating via a network, and communicating with various other systems, devices, processes, and information sources according to embodiments of the disclosure;

FIGS. 3A-F are views of a base station for the portable NEST headset;

FIGS. 4A-O are views of a portable NEST headset;

FIGS. 5A-E are views of a portable NEST base station and headset device;

FIGS. 6A-B are bottom views that depict examples of a portable headset device of the portable NEST system without and with a magnet cover according to embodiments of the disclosure;

FIG. 7 is an exploded view that depicts the portable headset device of the portable NEST system along with a base station of the potable NEST system according to embodiments of the disclosure;

FIGS. 8A-C are side views that depict examples of positioning of the portable headset device and positioning of magnets of the portable headset device with a user according to embodiments of the disclosure;

FIG. 9 is a diagrammatic view that depicts further examples of the portable headset device and its related systems and devices of an example portable NEST system according to embodiments of the disclosure;

FIG. 10 a perspective view that provides further detail of the portable headset device of FIG. 9 according to embodiments of the disclosure;

FIG. 11 is an exploded assembly view that depicts the portable headset device of FIGS. 9 and 10 according to embodiments of the disclosure;

FIG. 12 is a close-up diagrammatic view that depicts details of a NEST controller of the example portable NEST system of FIG. 9 according to embodiments of the disclosure; and

FIG. 13 is a diagrammatic view that depicts use of the NEST controller and the portable headset device of the example portable NEST system of FIGS. 9-12 according to embodiments of the disclosure.

### DETAILED DESCRIPTION

The disclosure relates to an electroencephalogram (EEG) and transcranial magnetic stimulation (TMS) process. In examples, the EEG and TMS process may be executed by any combination of a neuro-electroencephalogram synchronization therapy (NEST) system, an electrophysiology database and/or a TMS treatment system (optionally using an electrophysiology database and optionally providing for customized, patient-specific TMS treatment protocols, and/or optionally using a combined electrophysiology database and customized TMS treatment system), and/or any other computing device.

The portable NEST system may be easily carried or moved because the device or system may be lighter, compact, transportable, mobile, lightweight and/or smaller than typical or standard devices or systems. The portable NEST systems may include wearable NEST systems, NEST systems configured as headgear, positionable and removable NEST systems (e.g., may be conveniently positioned on or above a chair or seat and then easily removed from the chair or seat), modular NEST systems (e.g., capable of being integrated into another device or system), etc. The portable NEST system may be sized to have a maximum volume or a dimension range, such as about 285 mm length by about 194 mm width by about 188 mm height.

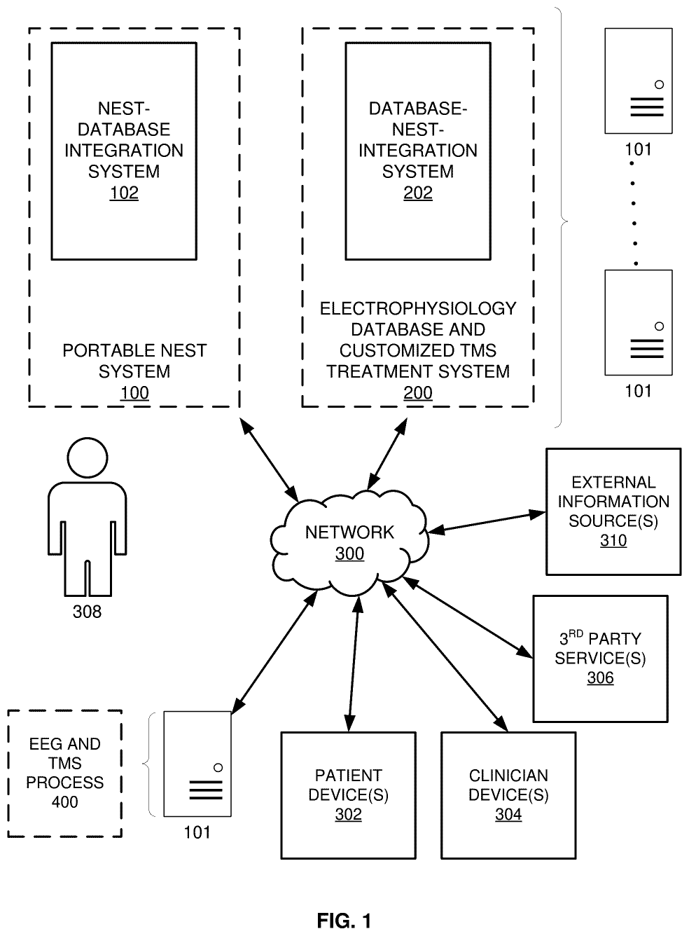

Turning now to FIG. 1 an example environment. The example environment includes a portable NEST system 100 with a NEST database (e.g., NEST-database integration system 102) in communication with a network 300. The network 300 is also in communication with an electrophysiology database and customized TMS treatment system 200 which includes a database integration system (e.g., Database-NEST integration system 202). One or more servers 101 can be provided in communication with the network 300 and/or components in communication with the network 300. Additional components in communication with the network 300 include, for example, external information source(s) 310, third party service(s) 306, clinician device(s) 304, and patient device(s) 302. An EEG and TMS process 400 may run across any combination of the systems and devices. The EEG and TMS process 400 may be run across any combination of an EEG system (such as a portable NEST system 100), an electrophysiology database and customized TMS treatment system 200, and/or another computing device (e.g., one or more servers 101 shown in FIG. 1). The EEG and TMS process 400 may be executed and run on the various systems and computing devices via a network 300. In FIG. 1, the portable NEST system 100 may communicate with the electrophysiology database and customized TMS treatment system 200 according to example embodiments of the disclosure. The portable NEST system 100 may use a NEST-database integration system 102 to communicate and interact with the electrophysiology database and customized TMS treatment system 200 via the network 300. Similarly, the electrophysiology database and customized TMS treatment system 200 may use a database integration system (e.g., Database-NEST integration system 202) to communicate and interact with the portable NEST system 100 via the network 300. Other devices (e.g., various computing devices or mobile devices) such as patient device(s) 302, clinician device(s) 304, and third-party service(s) 306 may communicate with the electrophysiology database and customized TMS treatment system 200 and the portable NEST system 100. The electrophysiology database and customized TMS treatment system 200 and the portable NEST system 100 may also communicate and interact with external information source(s) 310 via the network 300. As shown in FIG. 1, the portable NEST system 100 may be positioned on the head of a patient 308.

The portable NEST system 100 may capture EEG data from a patient 308. The electrophysiology database and customized TMS treatment system 200 may receive the EEG data from the portable NEST system 100. The electrophysiology database and customized TMS treatment system 200 may determine a TMS treatment based on the received EEG data. The portable NEST system 100 may provide synchronized transcranial magnetic stimulation (sTMS) based on the determined TMS treatment. The electrophysiology database and customized TMS treatment system 200 may also access the EEG data for a patient 308 and may classify the patient 308 as having a particular brain type based on the EEG data. The electrophysiology database and customized TMS treatment system 200 may determine a TMS treatment based on the classified brain type and the EEG data. The electrophysiology database and customized TMS treatment system 200 may recommend sTMS based on the determined TMS treatment. In some examples, the portable NEST system 100 may capture the EEG data from the patient 308 that may be accessed by the electrophysiology database and customized TMS treatment system 200. The portable NEST system 100 may provide the recommended synchronized transcranial magnetic stimulation to the patient 308. The electrophysiology database and customized TMS treatment system 200 may access the EEG data for a patient 308 and may analyze the EEG data. The analyzed EEG data can be used indicate brain health of the patient 308. The electrophysiology database and customized TMS treatment system 200 may determine TMS treatment based on the brain health indication and the EEG data. The electrophysiology database and customized TMS treatment system 200 may recommend sTMS based on the determined TMS treatment. In some examples, the electrophysiology database and customized TMS treatment system 200 may provide reporting of the brain health indication in the form of a brain health indicator for the patient 308. The electrophysiology database and customized TMS treatment system 200 may also use the brain health indication for diagnosing the patient 308. The electrophysiology database and customized TMS treatment system 200 may access the EEG data for a patient 308 and may provide bursting analysis of the EEG data. The electrophysiology database and customized TMS treatment system 200 may determine a TMS treatment based on the bursting analysis and the EEG data. The electrophysiology database and customized TMS treatment system 200 may also recommend sTMS based on the determined TMS treatment. Bursting analysis may be used for diagnosing the patient 308. The bursting analysis may be used for determining treatment parameters. The portable NEST system 100 may also capture EEG data from a patient 308 and have one or more rotating magnets for generating an alternating magnetic field. The portable NEST system 100 may determine a TMS treatment recommendation based on the EEG data or may receive the TMS treatment recommendation from a second system based on the EEG data. The portable NEST system 100 may provide sTMS by using the rotating magnets to generate the alternating magnetic field based on the determined TMS treatment or the recommended TMS treatment. The second system may be the electrophysiology database and customized TMS treatment system 200 for receiving the EEG data from the portable NEST system. The electrophysiology database and customized TMS treatment system 200 may determine the TMS treatment based on the received EEG data.

NEST System Components

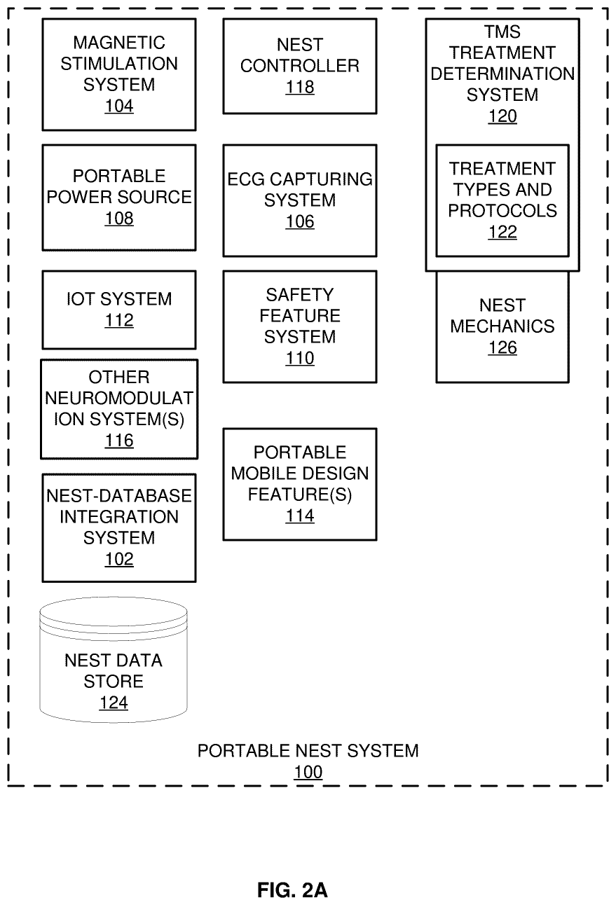

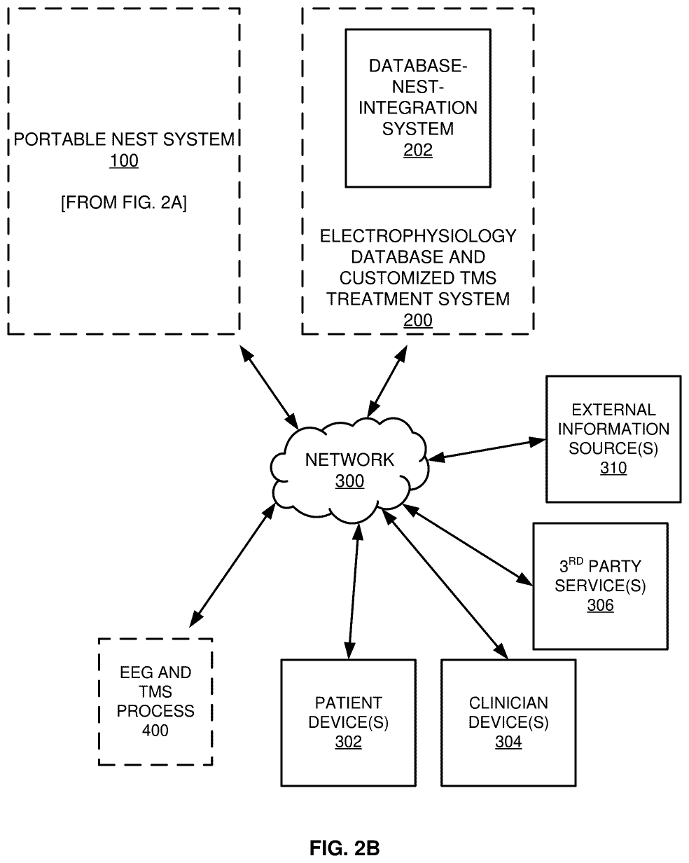

FIGS. 2A-B show a detailed view of the portable NEST system 100. The portable NEST system 100 may include the NEST-Database integration system 102 (e.g., portable NEST system software that enables integration with software of the electrophysiology database and customized TMS treatment system 200), a magnetic stimulation system 104 (e.g., including one or more magnets that may provide sTMS), an EEG capturing system 106 (e.g., detecting and capturing EEG data from the patient 308 using sensors such as electrodes placed along scalp of the patient 308), a portable power source 108 (e.g., portable-type power source such as a battery), a safety feature system 110 (e.g., one or more safety mechanisms that may prevent or avoid misuse of the portable NEST system 100 such as a locking mechanism), an Internet of Things (IoT) system 112 (e.g., including various components that may provide IoT capabilities such as relating to connecting the portable NEST system 100 to the network 300), portable/mobile design feature(s) 114 (e.g., one or more features that enable for portability and mobility use of the portable NEST system 100 such as miniaturizing of some features or components), other neuromodulation system(s) 116 (e.g., systems that provide other therapies such as light stimulation therapies), a NEST controller 118 (e.g., controller may be used for managing and directing one or more components of the portable NEST system 100), a TMS treatment determination system 120 (e.g., system for determining repetitive TMS (rTMS) and sTMS that may be based on the EEG data obtained by the EEG capturing system 106) having treatment types and protocols 122 (e.g., information and/or data related to treatment types and protocols as described in the disclosure that may be used by the TMS treatment determination system 120), a NEST data store 124 (e.g., storing any and all data related to use of the portable NEST system 100 and possibly some data received from other systems and/or devices such as received from the electrophysiology database and customized TMS treatment system 200), and NEST mechanics 126 (e.g., various mechanical parts relevant to configuration of the portable NEST device, such as an example portable headset device, for use by the patient 308). The network 300 can be connected to one or more patient device(s) 302, one or more clinician device(s) 304, third party service(s) 306, and external information source(s) 310. The headset device provides a support framework with attached electronic devices that is worn on the head of the user. The form factor of the headset can be a cap, helmet, or hat.

Portable NEST System Advantages

There may be several advantages to using the portable NEST system 100 over other traditional TMS devices such as wire or coil TMS devices. For example, one advantage may be favorable results (e.g., positive results in terms of sham effect) from feasibility tests or studies conducted with some example embodiments of the portable NEST system 100.

There may be other advantages to using the portable NEST system 100 compared to traditional TMS devices or systems. Traditionally, as described in the disclosure, TMS has typically used coils. A coil-based system has several disadvantages in that the coil requires a relatively large capacitor to be charged up and discharged through the coil. Frequently, there may be a thousand amps at a thousand volts passing through this coil that generate these pulsed currents in the brain. What has been found is that if treatment frequency (e.g., pulse frequency) may be configured to match a brain's intrinsic frequency, one can lower these levels of current and voltage. The portable NEST system 100 may use a routine permanent maintenance that may be a significant advantage over the coil devices in that 1) one can use the portable NEST system 100 continuously, and 2) the portable NEST system 100 may be less expensive.

Another advantage relating to the portable NEST system 100 may relate to the idea of energy at a resonant frequency. For example, a brain's intrinsic frequency may be at about ten Hertz. In some examples, if there is a pulsed signal (e.g., pulsed stimulus at about ten Hertz), it may be difficult to measure a pulse (e.g., the pulse may be detected at one point then undetected at a next point and then detected again, etc.). When looking at frequency distribution, the energy frequency domain may be quite wide and distributed. Whereas viewing the frequency distribution of a rotating permanent magnet (e.g., rotating magnets of the magnetic stimulation system 104 of the portable NEST system 100), it may be a pure sinusoid. Thus, most (if not all) of the energy of the portable NEST system 100 may be at the patient's intrinsic frequency.

Even though some coil devices may generate a power that may be significantly greater than the portable NEST system 100 (e.g., 100 times greater than the portable NEST system 100). When monitoring energy at a brain's resonant frequency, the portable NEST system 100 may have a higher energy. This may be due to most (if not all) of the energy from the portable NEST system 100 focusing on one frequency.

For providing magnetic stimulation of a brain of the patient 308 (shown in FIG. 1), the magnetic stimulation system 104 of the portable NEST system 100 may use a coil or a rotating magnet. Electrical stimulation may use, Transcranial alternating current (AC) stimulation. For example, an example magnetic stimulation system 104 may include electrodes that may be placed on the scalp or forehead of the patient 308 for providing an electric current (e.g., very low-level current). A very small portion of that current may be received in the brain. This may be a relatively small fraction of a total current that actually passes through a scalp of the patient 308.

Portability Designs

The portable NEST system 100 may have portability features (e.g., portable/mobile design features 114) in order to provide a portable NEST device. The portable NEST system 100 may be a NEST system that may have a portable unit device that provides sTMS. The portable NEST system 100 may be researched under sTMS. Suitable portable/mobile design features 114 include, but are not limited to, one or more of a specific range of dimensions and/or range of weights that allow for the portable NEST system 100 to have one or more of the example features, characteristics, and/or form factors relating to the portable/mobile design features 114. Portable/mobile design features 114 (e.g., dimensions) may be distinguishable from dimensions and/or other features of traditionally large TMS devices or systems that have been typically used. Various materials and/or components (e.g., having portable/mobile design features 114) may be included that enable a relatively small for integration into architecture of the portable NEST system 100.

The portable NEST system 100 may utilize portable in-home technology and mobile technology (for example, patients may take portable NEST system 100 from place to place). The portable NEST system 100 may also include portable/mobile design features 114 for enabling for portability and mobility use of the portable NEST system 100 such as by miniaturizing of some features or components. The portable/mobile design features 114 may make the portable NEST system 100 portable for use from home, or added to (e.g., part of) a transportation or vehicle system such as a car (e.g., using the portable NEST system 100 while driving or while a passenger of a vehicle as delivered by the installed portable NEST system 100), an airplane, a train, a boat, and/or other vehicle and other building establishments such as a hotel room, a restaurant, a retail store, and/or a user's place of business, other systems and establishments, and the like.

The portable NEST system 100 has a variety of applications including use in space travel to combat cognitive decline/reinforce wellness. For example, portable NEST system 100 may be designed and configured to be integrated into and/or added to (e.g., part of) a spacecraft, space station, and/or any other vehicles used in space as well as integrated into and/or added to any medical facility where astronauts may be treated after returning from space.

Portable/mobile design features 114 of the portable NEST system 100 enables the portable NEST system 100 to be shifted from a clinic-based technology (requiring physician oversight) to a remotely accessed used from any location outside the clinic (e.g., non-clinic settings such as home, office, etc.). The portable NEST system 100 may be used by patients for general wellness in-transit and/or from any location.

The portable/mobile design features 114 of the portable NEST system 100 may include design changes to a base station of the portable NEST system 100 so that each patient and/or clinician may hold the portable NEST system 100 in their hand or place the portable NEST system 100 in their lap or hold it in their hand. The portable NEST system 100 may be a hand-held device (e.g., slightly larger than a mobile device or a cell phone). The portable NEST system 100 has portable/mobile design features 114 that may allow for portable/mobile synchronous TMS treatment to be used to treat various disorders (e.g., attention deficit hyperactivity disorder (ADHD), post-traumatic stress disorder (PTSD), traumatic brain injury, etc.).

The portable/mobile design features 114 of the portable NEST system 100 may include design changes to a base station of the portable NEST system 100 relating to miniaturization of base medical technology to make it portable as described in the disclosure. The portable/mobile design features 114 may provide for a treatment arm that may be lowered onto a forehead where the patient 308 may lay on a massage table or bed and then the treatment arm may be lowered onto the patient's head. The portable/mobile design features 114 may allow for the treatment arm to be used in portable/mobile situations. The portable/mobile design features 114 may relate to a headset-shaped device. In this example, the NEST mechanics 126 along with the portable/mobile design features 114 may be designed to provide a single motor using a belt drive system in order to turn one or more magnets (e.g., three magnets of the magnetic stimulation system 104). This example headset-based system may include three individual motors (e.g., small, sophisticated motors similar to radio-controlled (RC) car motors) that rotate the magnets each of them at a same frequency. These parts and components of the magnetic stimulation system 104 (e.g., magnets), the NEST mechanics 126 (e.g., motors), and portable/mobile design features 114 (e.g., relating to design features/components that may be miniaturized in order to make the headset-based device mobile). For example, portable/mobile design features 114 may allow for the base station and/or controller (e.g., the NEST controller 118) with a cable that may be positioned to the headset-base device. The base station may be designed so that a person or patient may hold the headset-based device in their hand or set it in their lap.

The portable NEST system 100 may include a neuro-EEG synchronization therapy system having a portable unit device that may provide sTMS. The portable unit device may include the portable/mobile design features 114 such as a mobile design developed through miniaturization and compacting of components within the portable unit device (e.g., of the portable NEST system 100) allowing for use at various locations and when the patient is in-transit between locations. In examples, the portable unit device (of the portable NEST system 100) may be configured as a wearable headgear that provides the TMS treatment during mobility of the patient.

Portable Power Source

The portable NEST system 100 may also include a portable power supply to power the system. A suitable portable power source 108 is a battery. The portable power source 108 may be designed to be operated and self-contained thereby not requiring an external power source. The portable power source 108 may be rechargeable in some configuration thereby allowing the portable power source 108 to be plugged into an electrical outlet. As will be appreciated by those skilled in the art, use of the portable power source 108 may avoid the typical need to charge a capacitor to extremely high voltages and that may be discharged via the coil. Some of these conventional TMS processes of charging the capacitor and discharging the coil may not work with batteries. Also, some of these conventional TMS systems may need or require a special breaker installed in the electrical system of an office in order to run the device for safety purposes because these conventional TMS systems or devices may typically draw a relatively large amount of power. In contrast, the portable NEST system 100 may draw a relatively small amount of power (compared to conventional TMS systems) which enables the portable NEST system 100 to function as a battery-operated device. Additionally, the portable NEST system 100 may run for several hours from the portable power source 108 without a recharge and may be recharged as needed. The portable power source 108 (battery) may be positioned inside or in communication with the NEST controller 118 which may be plugged into an electrical outlet to recharge. Alternatively, the portable NEST system 100 may utilize wireless charging to recharge the portable power source 108. For example, recharging of the battery may be performed through Wireless Power Transfer (WPT) (or wireless power transmission or wireless energy transmission (WET) or electromagnetic power transfer), in which an external energy source couples with the battery in order to recharge the battery. In one example, the external power source may be a radio frequency (RF) antenna that transmits power wirelessly. The external power source may be an electromagnetic coupling mechanism, such as a coil, that couples with the battery in order to provide power to recharge the battery. The portable power source 108 may be recharged through inductive charging (e.g., utilizing similar inductive technology as used with mobile devices such as smartphones). Treatment may be provided by the portable NEST system 100 while the battery may be plugged into electrical outlet if compliance allows for such use. Typically, for compliance, if a device used on a patient is plugged into an electrical outlet, there needs to be medical-grade power supplies and other things. Alternatively, as the portable NEST system 100 allows for treatment of patients through the portable power source 108, compliance may be followed more easily as there may be little to no risk to patients from getting shocked by the main power supply.

Design Features Based on Patient Category

The portable NEST system 100 may include a portable unit device that may provide sTMS where the portable unit device has design features (e.g., portable/mobile design features 114) based on patient category. Patient categories may be based on demographics (e.g., age of patients), patient physical characteristics (e.g., head size), patient-related activities, and/or mental disorders of patients. Different patient populations may have different requirements. Patients with post-traumatic stress may, for example, have different general needs as these patients may tend to be younger and more mobile. Thus, in some examples, the portable NEST system 100 may be worn while a person is moving around as opposed to staying in one place. The portable NEST system 100 may be used for dementia and for Alzheimer's, in which case the mobility requirements may be different from these other mental disorders. The portable NEST system 100 may also be used in conjunction with other therapies, such as speech or motor-related therapies. The portable NEST system 100 may be used in athletic or competitive training, such as during fine-motor tasks or physical activity.

For improving performance in some activity, the design of the portable NEST system 100 (e.g., portable/mobile design features 114) for those individuals may be more technology heavy. The design may be edgy such that the portable NEST system 100 may use smart glasses (e.g., Google® Glass). The portable NEST system 100 may be integrated with other digital applications or hardware for addressing different performance outcomes. Whereas treatment of older populations (e.g., especially for some of the conditions such as mild cognitive impairment, dementia, and Alzheimer's), the design may have features that may provide a comfortable feel. For post-traumatic stress, the portable NEST system 100 may be technological.

Headset and Base Station Configurations

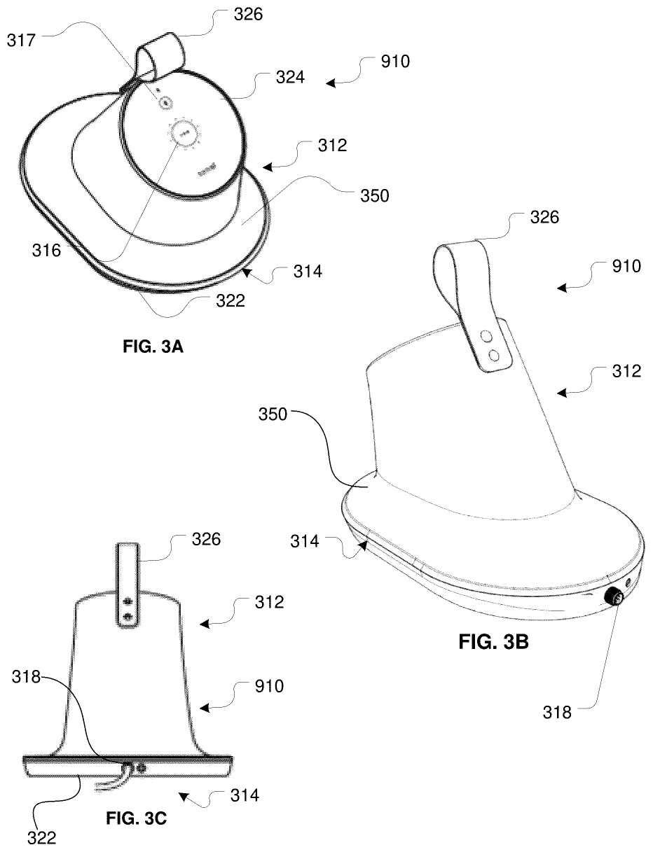

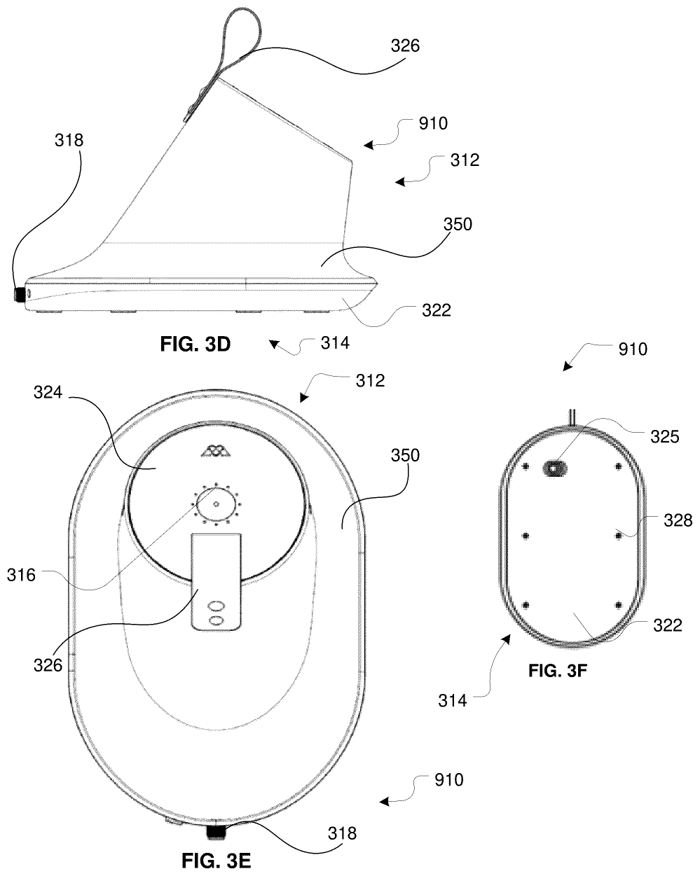

FIGS. 3A-F are views of a base unit 910 or base station for the portable NEST headset. FIG. 3A is perspective front view of the base unit 910, FIG. 3B is a perspective back view of the base station 910, FIG. 3C is a front view of the base station 910, FIG. 3D is a side view of the base station 910, FIG. 3E is a top view of the base station 910, and FIG. 3F is a bottom view of the base station 910. The base station 910 has a housing with an upper portion 312 and a lower portion 314. The diameter across the bottom surface 322 of the housing, as illustrated and appreciated in FIG. 3F, has a rectangular shape with curved edges and a diameter that is wider than a diameter across an upper surface 324. The bottom surface 322 can have an on-off switch 325, and a plurality of pads or feet 328 which raise the base station 910 from coming into flush contact with a surface. A cable connection 318 is also provided. The base station can have a therapy controller 316, and a Bluetooth activator 317. The therapy controller 316 starts, pauses, or hard resets a therapy session. The upper surface 324 has a strap 326 or handle usable to pick-up the base station 910 or base station in combination with the headset. The handle or strap 326 can be removably or permanently secured to, for example, a side of the upper portion 312 of the housing by one or two mechanical connectors. Suitable connectors include snaps and rivets. The housing has an exterior surface and an interior surface operable to hold the device electronics. The upper portion 312 of the housing features a sloped side wall and an upper surface 324 that is not parallel to the bottom surface 322. The upper surface 324 has an angular columnar feature that extends upward and is operable to engage the headset.

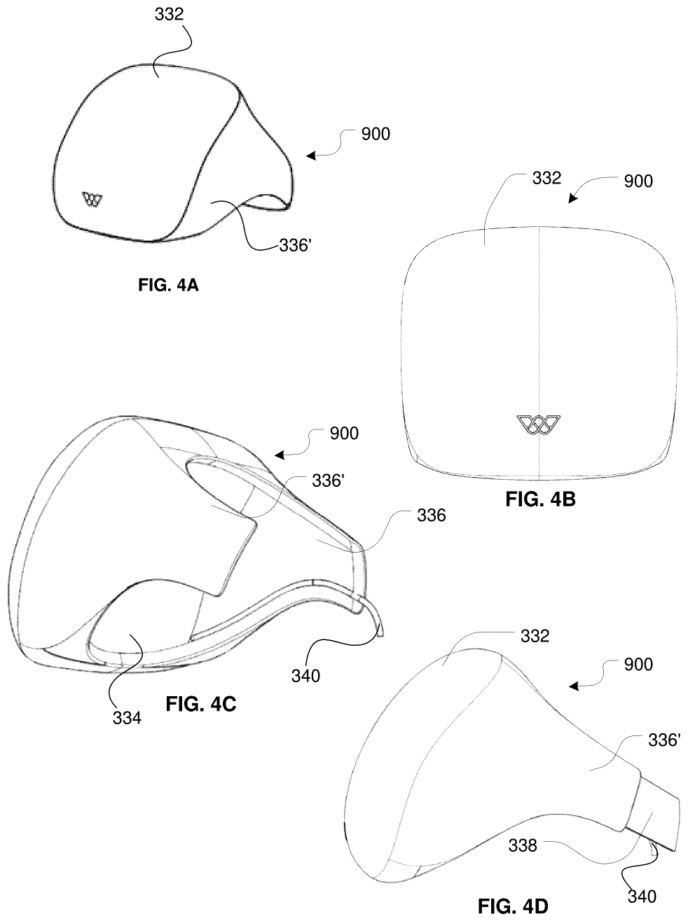

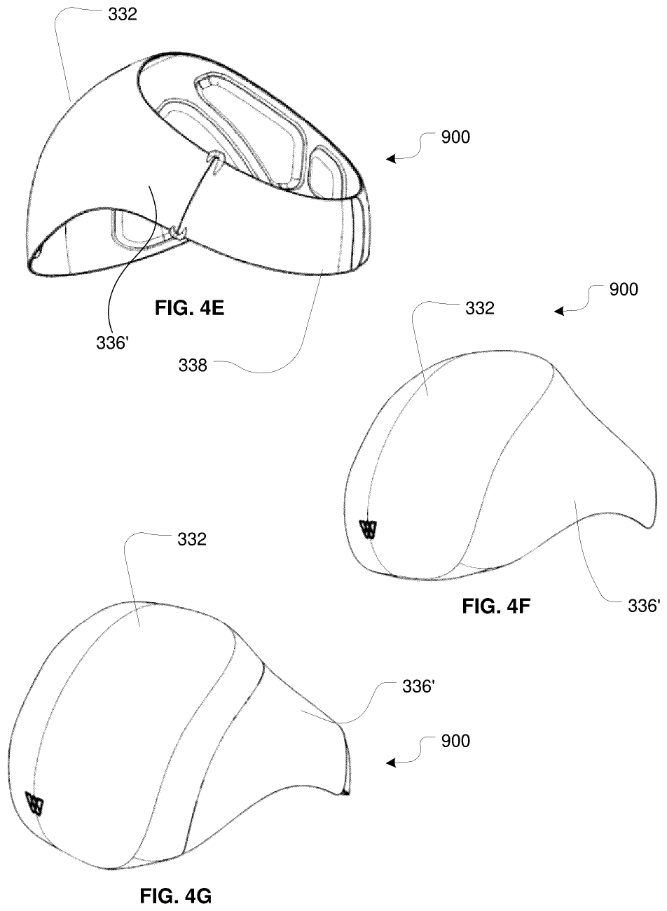

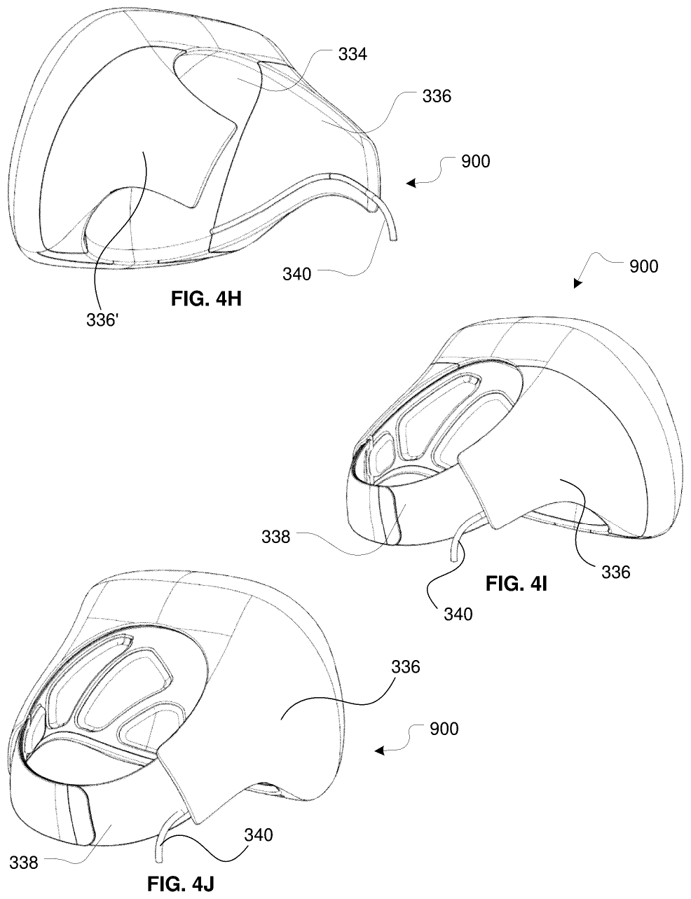

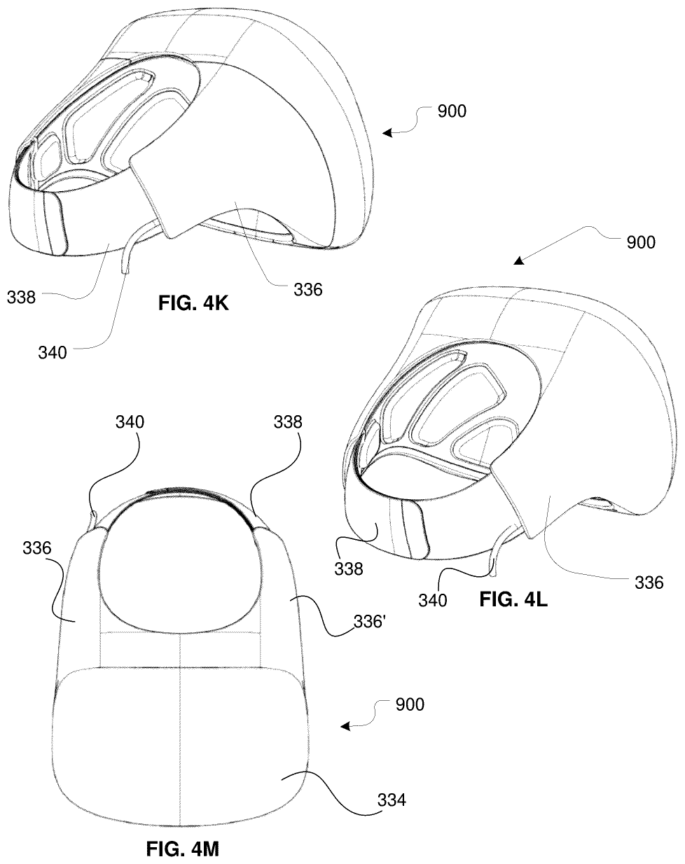

Turning to FIGS. 4A-O, views of a portable NEST headset 900 are illustrated. FIG. 4A is perspective front view of the headset 900, FIG. 4B is a front view of the of the headset 900, FIG. 4C is a perspective back view of the of the headset 900, FIG. 4D is a side view of the headset 900, FIG. 4E is a perspective back view of the headset 900 with the strap attached, FIGS. 4F-G are perspective front views of the headset 900, FIG. 4H-L are perspective back view of the headset 900 with and without the strap, FIG. 4M is a top view of the headset 900 with the strap, and FIGS. 4N-O are views of the headset 900 with and without the magnet cover. The headset 900 has a smooth front surface 332 which is curved, and a smooth interior surface 334 which has a convex curved shape to accommodate a user's head. A pair of side members 336 are provided which are sized and configured to be positioned against the side of a user's head. A strap 338 is provided which connects the first side member 336 to the second side member 336′. A cable 340 can be provided that engages the base station. Electronics controlling operation of the device and magnets for delivery of therapy are positioned within the headset housing. FIGS. 4N-O illustrate an interior surface of the headset 900 with and without the magnet cover 904. The magnet cover 904 includes one or more padded surfaces to increase comfort for the user when wearing the headset 900.

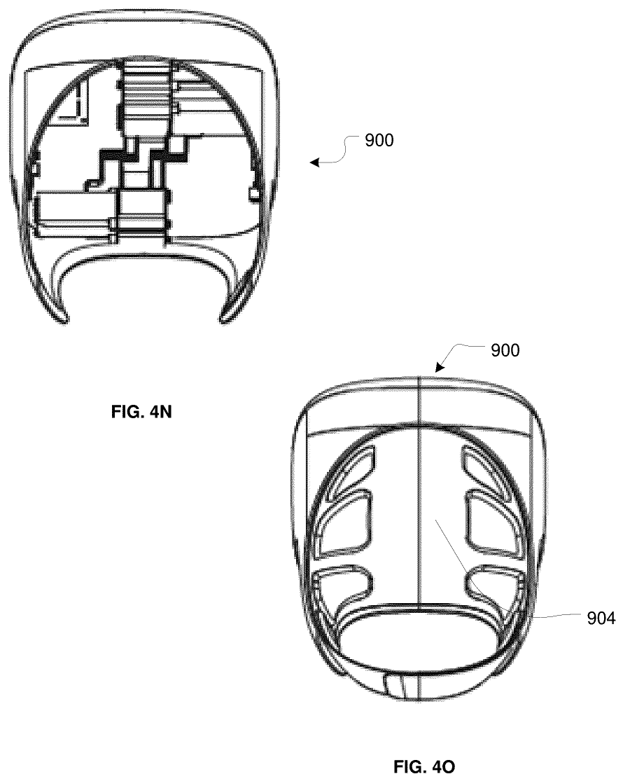

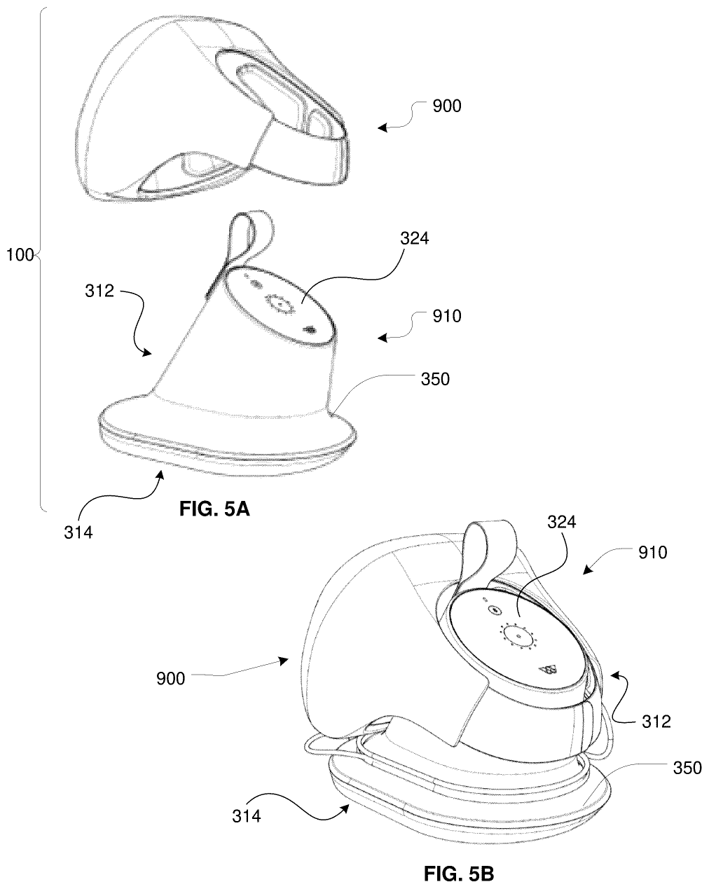

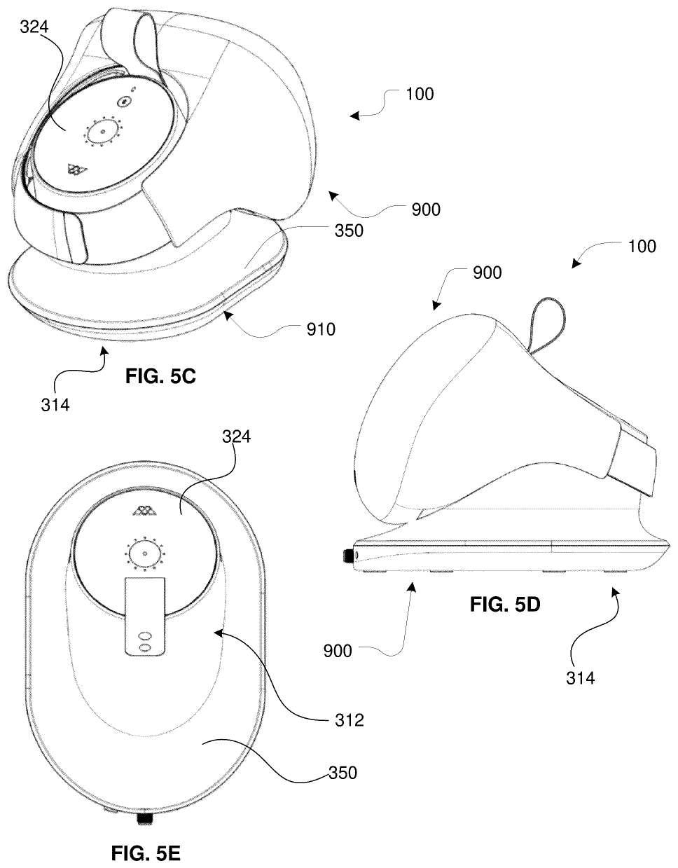

FIGS. 5A-E are views of a base station 910 and the headset device 900 of the NEST system 100. The headset fits over the upper portion 312 of the base station housing when the device is not in use. The base station strap expends upward and is accessible by the user to allow for easy carrying. The angled round shape of the upper portion 312 and the broader diameter of the lower portion 314 of the housing allow the headset to sit snugly in storage with the front portion of the headset resting on a widened lip 350 formed as part of the upper surface 324 of the housing.

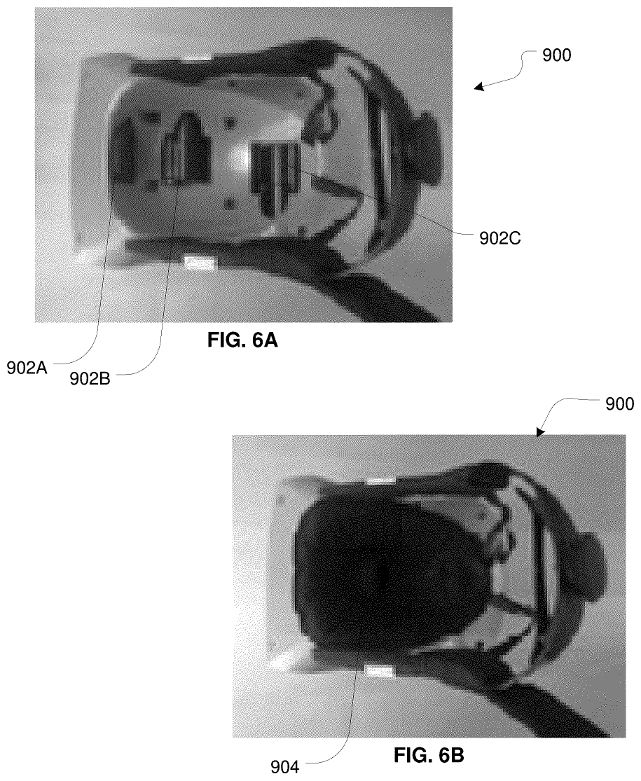

FIGS. 6A-B illustrate a portable headset device 900 of the portable NEST system including three magnets 902A, 902B, 902C as shown with (FIG. 6B) and without (FIG. 6A) a cover (e.g., magnet cover 904) for the magnets 902A, 902B, 902C.

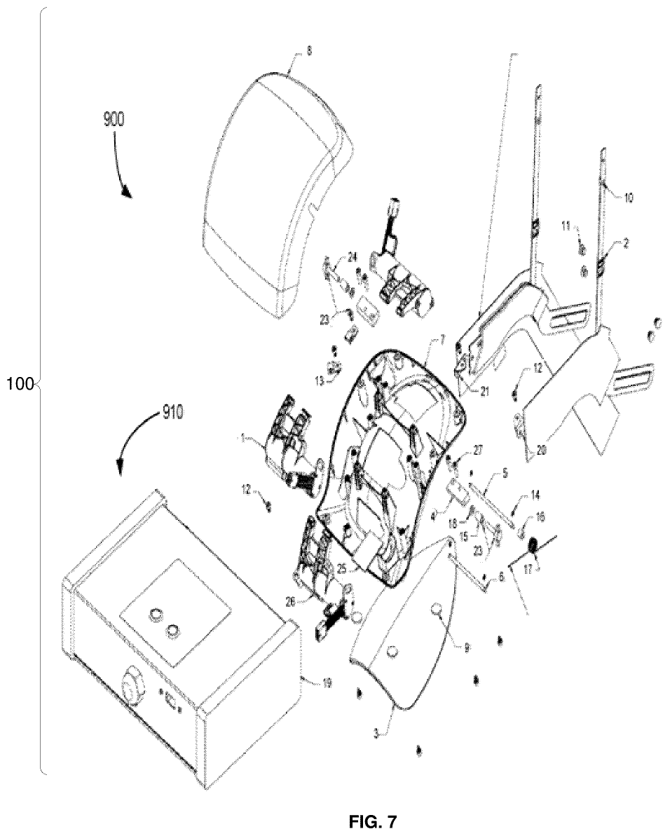

FIG. 7 illustrates a portable NEST system 100 and many of its components included in the exploded assembly view of the portable headset device 900 along with a base station 910 usable for controlling the portable headset device 900. Components may include: adjustable motor magnet housing assembly 1, headband assembly 2, liner pad assembly 3, adjustment block 4, pivot rod (pivot rod top 5 and pivot rod bottom 6), lower shell 7, upper shell 8, ⅝″ Velcro dot 9, locking hammer set snap 10, rivet headband mount 11, M4×6 socket head cap screw (SCHS) 12, cable tie holder 13, snap ring 14, ½ ″ #10 Garolite spacer 15, M4×8 spacer nylon 16, 1.47 in-lbs torsion spring 17, stainless steel M5 washer 18, electronic enclosure assembly 19 (may also be referred to as base station 910), left headband bracket 20, right headband bracket 21, 6 in cable tie (not shown), DP 100 epoxy 23, 10-32, 1″ plastic head thumb screw 24, fixed motor vibration dampening foam cut 25, fixed motor magnet housing assembly 26, and M4×14 SCHS 27. In this example, the portable headset device 900 may also include a cable extending from the electronics enclosure assembly 19 or the base station 910. Also, in this example, epoxy may be applied to form a bead around the entire joint between screw head and spacer (e.g., head thumb screw 24 and spacer nylon 16).

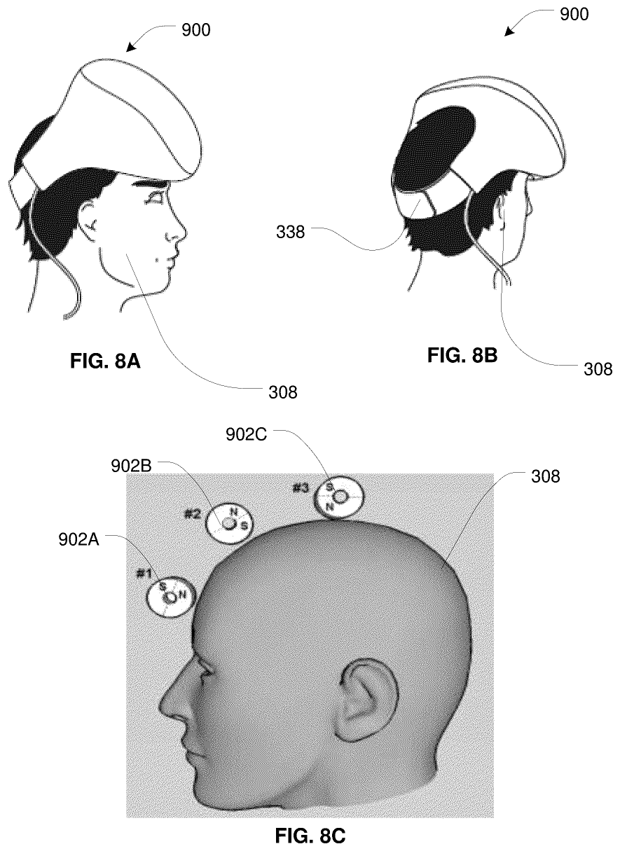

FIGS. 8A-C illustrate use of a portable headset device 900 and positioning of the rotating magnets 902A, 902B, 902C with a patient 308. As shown in the figure the orientation of the magnet poles (North and South) are slightly rotated from magnet 1 902A to magnet 2 902B to magnet 3 902C. Moreover, the rotating magnets 902A, 902B, 902C are positioned along the sagittal line above the patient's scalp, which rotates along a transverse axis.

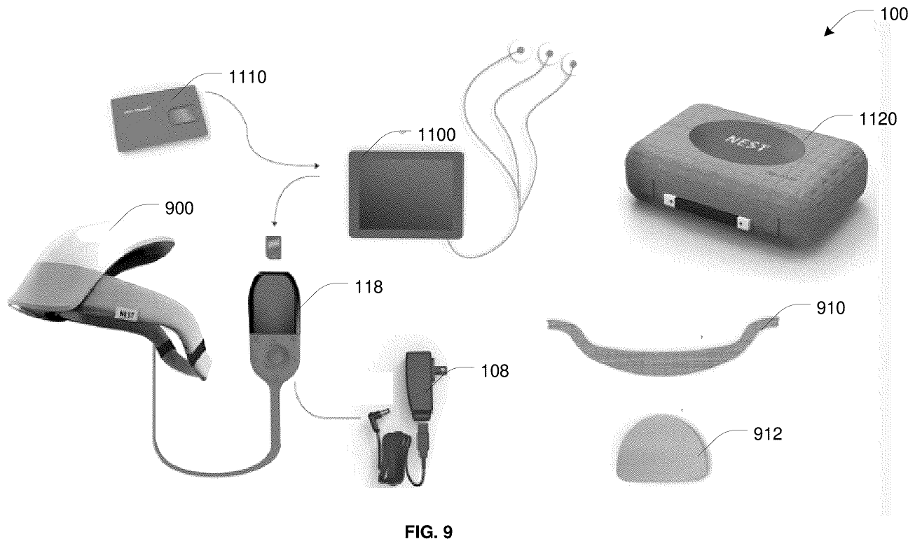

FIG. 9 illustrates components of a portable NEST system 100 including components that may be part of a kit and may include the portable headset device 900, the NEST controller 118, the portable power source 108, an IAF programmer 1100, a subscriber identify module (SIM) card 1110, a protective case 1120, a headband 910 and a liner pad 912.

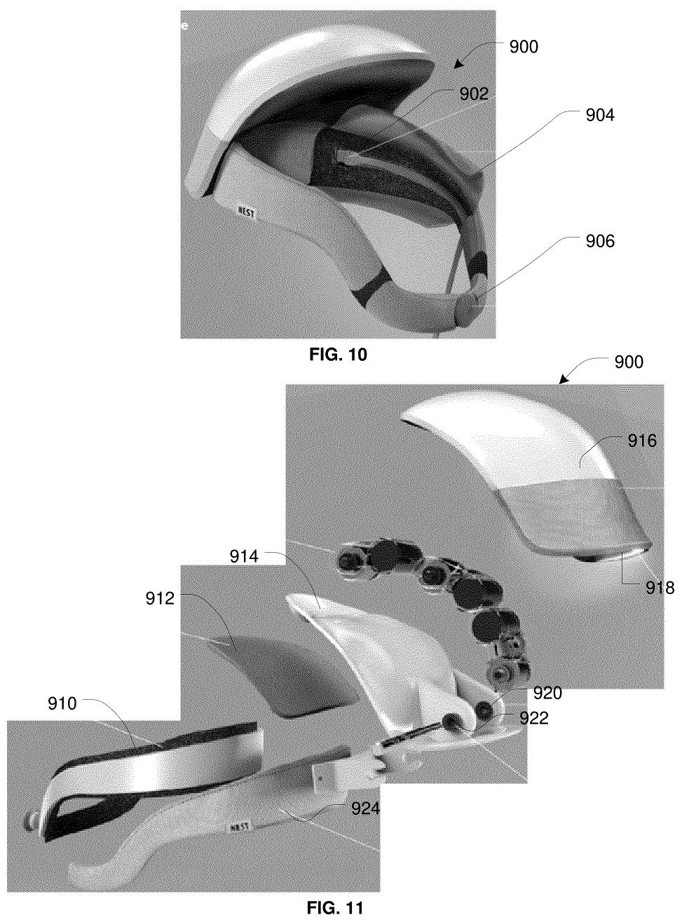

FIGS. 10 and 11 illustrate examples of a portable headset device 900 of the portable NEST system 100. The portable headset device 900 has a connection to a controller cable 902, a headband wrap 904 (which can be a consumable), and an adjustment knob 906 to facilitate fitting the device during use. FIG. 11 is an exploded view of this portable headset device 900 in FIG. 10. The exploded view further illustrates a removable headband 910, a head liner pad 912, a magnet and motor housing 914, an upper head mounted device housing 916, one or more LEDs 918, a lower head-mounted device housing 920, a motor/magnet array pivot 922 and a padded headband wrap 924.

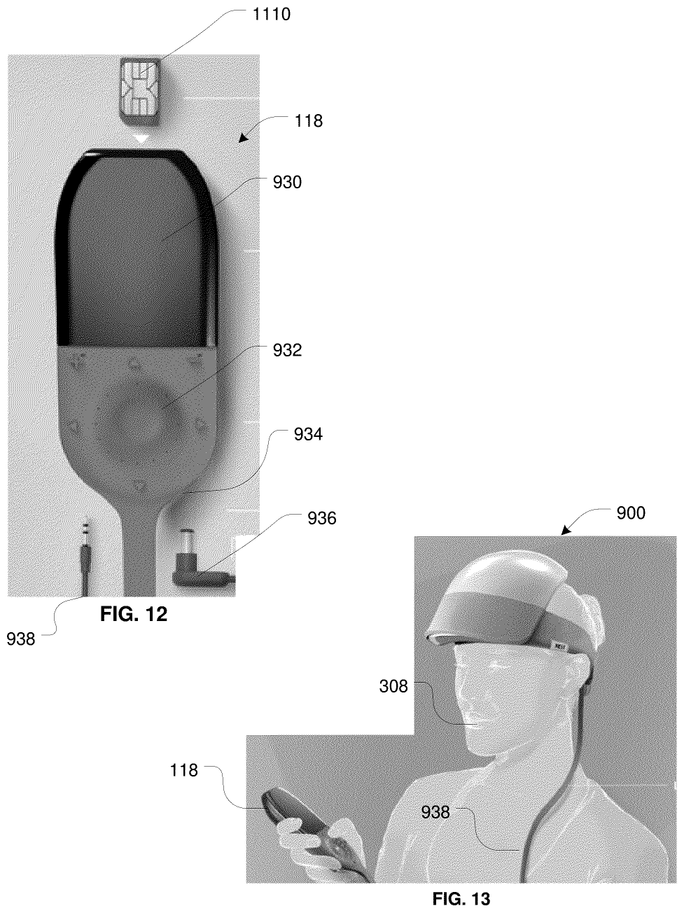

FIG. 12 illustrates a detailed view of the NEST controller 118 of the portable NEST system 100. The NEST controller 118 may include a screen interface 930, a button array 932, an optional headphone jack 934, and an AC adapter 936. Additionally, the NEST controller 118 may receive the SIM card 1110 or a PPM, as shown in FIG. 12. The NEST controller 118 may use a wireless connection (e.g., Bluetooth) with a network to accelerate data transfer in a cloud database.

FIG. 13 illustrates use of the example portable NEST system 100 (e.g., as shown in FIG. 9). FIG. 13 further details use of the NEST controller 118 and the portable headset device 900 of the portable NEST system 100 with the patient 308 (e.g. patient user). As illustrated the cable 938 between the NEST controller 118 and the portable headset device 900 lays flat against the user.

Headset Design Imparts Stimulation to Precise Location

The portable NEST system 100 may include a NEST system having a portable unit device that may provide sTMS that may relate to defined treatment types and protocols (e.g., using the treatment types and protocols 122 of the portable NEST system 100 and/or the treatment types and protocols of the electrophysiology database and customized TMS treatment system 200 described herein) such that the device may provide sTMS at precise pulse frequency values and/or physical locations. In some examples, the sTMS may relate to a defined treatment protocol. The portable NEST system 100 may include a technologically advanced headset design that may impart stimulation at a precise pulse frequency values and/or precise physical locations. Treatment may be at a specific frequency level that the brain may be most comfortable with in terms of operation. Geo-navigation may be used to navigate to an area of the brain that may need stimulation and may give precision guided treatment to that location of the brain. In some examples, deep brain regions of the brain may be targeted.

The magnetic stimulation system 104 of the portable NEST system 100 may comprise a plurality of permanent magnets (e.g., three magnets as shown in FIGS. 8 and 11); the number of magnets may be increased as needed to accomplish specific therapeutic applications. The portable NEST system 100 may also be used to improve the rhythmicity of neuro-firing in the brain. The portable NEST system 100 may be used to try to make the brain firing mode more coherent, more regular which may address issues with individuals who may have a mental disorder or other problems, e.g., as part of the EEG and TMS process 400, with access to EEG data of the patient 308 (acquired from the EEG capturing system 106), mental disorders may be discovered where EEG data may be chaotic, more distributed than typical EEG data, and energy of firing may be distributed widely throughout a spectrum. The portable NEST system 100 may be used with patients to increase general wideband activity (e.g., increase beta activity or gamma activity) which may be related to (e.g., correlated with) performance/awareness for patients and may facilitate improvement of learning for patients.

The EEG and TMS process 400 may utilize a procedure that indirectly stimulates larger regions of cortical tissue or provides global stimulation of cortical tissue where frequency may be targeted as opposed to a location. There may be specific locations in the brain that may be targeted because a magnetic field drops off as the distance from the source of the magnetic field increases (e.g., drop by about one-third). Three centimeters away from rotating magnets, an amount of magnetic field in accord may be extremely small. Accordingly, the EEG and TMS process 400 using the magnetic stimulation system 104 may mainly affect areas directly underneath magnets such that the brain may use entrainment in which neurons may affect neighboring neurons. Thus, use of the portable NEST system 100 may result in a distributed phenomenon where the area under the magnet may be made very rhythmic and this rhythmic firing may affect the firing of other neurons. This may be a field effect. For example, stimulation may target highly connected circuits, such that stimulation of one end of the cortical circuit results in the stimulation of the other end (e.g., such as nigro-striatal or thalamo-cortical or temporo-parietal circuits). When a neuron fires, the neuron may generate a small electromagnetic field around it, which may then affect the firing of adjacent neurons. Additionally, the portable NEST system 100 may influence a local field potential (LFP), which may locally stimulate tissue and may propagate to adjacent tissue via LFP. It may be preferred to affect specific areas directly such as a prefrontal cortex. This area may be generally associated with mood and higher-level processing. The prefrontal cortex may be typically located underneath the forehead of the patient 308. The left dorsolateral prefrontal cortex may also be targeted which is over the left region of a person's forehead. This area may be traditionally targeted by TMS devices. TMS devices may frequently place a coil above the forehead for targeting mood. Other areas in the brain may be targeted such that magnets of the magnetic stimulation system 104 may be shifted around one or more targets. The magnetic stimulation system 104 may target the motor cortex in order to help with essential tremor or other diagnoses or the magnetic stimulation system 104 may target an occipital lobe for addressing vision issues. The magnetic stimulation system 104 may include any number of magnets that may be packed reasonably into the device with minimal disruption to quality of performance of magnets (e.g., due to magnets having some effect on each other). For example, when the magnets (e.g., high-power permanent magnets) rotate, the magnets may influence each other as they rotate. Thus, if magnets are too close together, the motors (e.g., of the magnetic stimulation system 104 or NEST mechanics 126) may not be powerful enough to rotate the magnets as the proper frequency or phase, and stimulation, may be untenable due to closeness of distance between these motors and magnets.

The EEG and TMS process 400 may include determining a location for treatment and/or for adjusting location of treatment as being customized for each person. The EEG and TMS process 400 may utilize the portable NEST system 100 and the electrophysiology database and customized TMS treatment system 200 to accomplish this customized location treatment. The EEG and TMS process 400 may be configured to determine the patient-specific location based on how TMS may be affecting each patient and then may change location of TMS treatment. A task may also be performed and based on a region activated in the cortical tissue, the location being treated and/or other parameters of the stimulation may be adjusted accordingly. A suitable task can include a subject practicing fine motor skills in a surgical technique simulation exercise while EEG activity are recorded. Tissue exhibiting novel EEG activity may only be present during the task in a band of interest, for example gamma, is observed, and that tissue may then be selected as the stimulation site. A subject reading course material for an academic class can have EEG activity monitored for novel EEG activity that is only present during reading. That information may then be used as the stimulation site once identified. Similarly, this may be done for frequency where a task may be performed that may result in a specifically generated frequency (e.g., in various ranges including one or more of an alpha range, beta range, delta range, theta range, gamma range, etc.) and then a corresponding stimulation may be adjusted in terms of location and frequency of treatment and/or other parameters of the stimulation (e.g., adjust TMS treatment). In some examples, the magnets of the magnetic stimulation system 104 may be mounted in a fixed position. The magnetic stimulation system 104 may be used to determine and select treatment locations based on each patient. The magnetic stimulation system 104 may be used to shift magnets towards a tailored location specific to each patient. For example, from looking at a patient's EEG, the EEG data may not be uniform throughout their brain. Referring to chaotic activity, the chaotic activity may be focused in particular areas. In some examples, the portable NEST system 100 may use the magnetic stimulation system to shift and/or adjust magnets' location to where there may be chaos. The portable NEST system 100 may be configured as a small device that may be easy to use such that magnets may be shifted around based on the patient's EEG data or based on a patient preference. The EEG and TMS process 400 may utilize the portable NEST system 100 (e.g., specifically the TMS treatment determination system 120) to locate specific positions that may be more effective than other positions for the patient 308 based on their feedback. In general, the portable NEST system 100 may be designed to be small, easy-to-use while having the capability of shifting magnets around based on patient's EEG data and/or based on a patient's preference or performance in a specific task or based on symptoms of a mental disorder.

Environments and Variety of Uses

The portable NEST system 100 may be used in various possible ways in applying TMS (e.g., rTMS and/or sTMS). The portable NEST system 100 (e.g., as a portable unit device) may be integrated and used in various user environments. The portable NEST system 100 (e.g., battery powered NEST device) may be configured to be used in different environments such as at home, while watching television, while meditating, in bed, in a car, etc. The portable NEST system 100 may also provide stimulation to the brain in order to make it produce the optimal variety of brainwaves for aiding performance in whatever activity a patient may likely be performing. The portable NEST system 100 improves relaxation on airplanes, focus while driving, or any number of mental states for a specified task or activity. Over the course of the 24-hour day, the patient's brain may keep various brainwaves active (e.g., typically within five bands—within delta, theta, alpha, beta, gamma bands). Delta and theta activity may be most generally present during shallow and deep stages of sleep, depending on sleep stage. Alpha, beta, and gamma activities may be most generally dominant in the EEG during wakefulness. Alpha activity may be generally present during states of reflection and during eyes closed periods. Beta activity may be present during waking, active states. Gamma activity may be generally present during task learning and acquisition, and in contrast to other EEG activities generally only appears in local tissue and disperse between cell layers. Depending on what the patient may be doing, some brainwaves may be more active in certain areas of the brain while other brain waves may be less active in other areas of the brain, while activity within these brainwave bands may not be fully “switched off” per se. In specific examples, the portable NEST system 100 may be used in first-class airlines seats of jets, used while sleeping at night, and/or used while undergoing a test. The portable NEST system 100 may be used while performing an activity requiring focus and/or concentration such as used with a race car driver, a truck driver, etc. The portable NEST system 100 may also be used in virtual reality environments (e.g., utilizing virtual reality technologies).

There may be other ways of using the portable NEST system 100. The portable NEST system 100 may include the patient 308 receiving stimulation while the patient 308 may be undergoing a test or an activity. While the stimulation may be occurring, the patient 308 may get significant benefits in focus. In some examples, the portable NEST system 100 may be used by the patient 308 while the patient is performing activities. For example, a racecar driver may wear the portable NEST system 100 while driving or immediately prior to driving the racecar in order to improve their focus and concentration so that the driver may not drift and improve ability in trying to accomplish their goal. Another similar example use of the portable NEST system 100 may be with a truck driver that may have long distance travel. The portable NEST system 100 may help the truck driver focus with long hours thus avoiding and/or lowering risks of accidents. For example, if the truck driver may sense that they are drifting off or sleepy, they may stop their truck for a break and use the portable NEST system 100 to help improve their focus going forward, or the portable NEST system may detect drowsiness and automatically begin stimulation in order to reinforce focus. The portable NEST system 100 may be used on-demand by various people. The portable NEST system 100 may be used during all or only part of an activity. The portable NEST system 100, when used during a test, may only be required either when improved focus is required or when the patient 308 has attention that is flagging. The portable NEST system may be activated or deactivated by the patient 308 on-demand, or may be activated or deactivated automatically by detecting a change in a mental state of the patient 308.

Patients may use the portable NEST system 100 in combination with other modalities. The portable NEST system 100 may also be used along with other stimulation that might augment entrainment effects, such as through rhythmic stimulation as sound, lights, other stimulations, and/or other therapies.

The portable NEST system may be used with virtual reality (e.g., used with a virtual reality device such as virtual reality goggles). There may be various activities that patients may engage in while undergoing stimulation using virtual reality. The portable NEST system 100 may be a separate device, which is worn in conjunction with or integrated with the virtual reality device. The portable NEST system 100 may incorporate virtual reality capabilities such that the patient 308 may only need to operate a single device to obtain the benefits of both virtual reality and the NEST therapy.

The portable NEST system 100 may be used in different situations or different scenarios. The portable NEST system 100 may be used at a storefront at the mall (e.g., people may go in for a regular back massage and they may obtain a brain massage with the portable NEST system 100). For example, a person may feel that they are having trouble concentrating or may not feel well and the person may stop at a store for twenty minutes of NEST treatment in order to improve their mental health. The EEG and TMS process 400 may include use of the portable NEST system 100 combined with a massage or may be combined with other relaxation treatments (e.g., sensory deprivation chamber). The portable NEST system 100 may be used at home, during sports events, and similar activities.

The portable NEST system 100 may be used with patients when they sleep or as they fall asleep. Use of the portable NEST system 100 may improve sleep. For example, brains of absorbers (e.g., rhythmic brains) may tend to have trouble sleeping (e.g., have insomnia) whereas high-energy brains (e.g., emitter-type of personality) may have an easier time sleeping (e.g., greater ease in falling asleep). By shifting a patient's brain to a less rhythmic state or higher energy state, the treatment by the portable NEST system 100 may help with focus and may help with sleep.

The EEG and TMS process 400 and specifically the portable NEST system 100 may be used to help patients fall asleep, achieve deeper sleep states, and treat patients when they are sleeping. For some people, there may be feedback from running stimulations such that they may find that the TMS treatment may disrupt their sleep because the stimulation may accentuate their normal resting awake brain waves as opposed to frequencies for sleep. However, the feedback with sleep may be resolved by varying frequency of the rotating magnets of the magnetic stimulation system 104 such that instead of accentuating intrinsic alpha frequency, the magnetic stimulation system 104 may reinforce delta and theta bands which may be typically observed during stages 2 and 3 of sleep. Additionally, stimulation may move into upper alpha and lower beta frequency ranges during REM sleep, to facilitate or augment resultant dreaming. The stimulation frequency may be modeled to follow the individual's sleep cycle and EEG rhythms specific to the patient corresponding to each stage of sleep. The EEG and TMS process 400 may use the portable NEST system 100 to record EEGs (e.g., using the EEG capturing system 106) during sleep and then may gradually adjust magnetic fields to accentuate various frequencies (e.g., reinforce delta and theta bands during stages 2 and 3 of sleep and move into upper alpha and lower beta frequency ranges during REM sleep) that may be used to bring patient to, for example, rapid eye movement (REM) sleep or deep sleep. The EEG system may indicate when the subject is transitioning between states, and then stimulation may be used to encourage observed transition. As stimulation is subthreshold, patients may not feel the magnetic field (e.g., do not feel electric currents generated) from the portable NEST system 100. As described in the disclosure, electric currents may be relatively small with the portable NEST system 100. In contrast, conventional rTMS devices with a coil may sit above the forehead and may apply stimulation that may not be conducive to sleep.

Neuromodulation with Stimulating Forces (e.g., Magnetic Stimulating Forces) Delivery

The portable NEST system 100 may include a neuro-EEG synchronization therapy system having a portable unit device that may provide sTMS that may relate to treatment types and protocols (e.g., using the treatment types and protocols 122 of the portable NEST system 100 and/or the treatment types and protocols 214 of the electrophysiology database and customized TMS treatment system 200 described in the disclosure) such that the sTMS may include magnetic stimulating forces that may provide neuromodulation. The portable NEST system 100 may use the magnetic stimulation system 104 to provide neuromodulation with stimulation forces (e.g., magnetic stimulation forces). The portable NEST system 100 may use the magnetic stimulation system 104 to apply a magnetic force in cortical tissue and then may change or influence how that cortical tissue may behave. The portable NEST system 100 may apply one force as magnetic stimulation that may be generated at a specific frequency. The portable NEST system 100 may apply overlapping forces. The portable NEST system 100 can also apply multiple overlapping frequencies, or different frequencies from each rotating magnet. For this example, the portable NEST system 100 may apply the same multiple magnetic pulses in different locations or a combination of forces. The portable NEST system 100 may use the same focal magnetic pulses in different locations or a combination of forces.

With magnetic stimulation, the EEG and TMS process 400 may also calculate the intrinsic frequency (e.g., using algorithms). The EEG and TMS process 400 may identify a frequency where the energy of the EEG may be at its maximum. This may be how an optimal frequency setting may be determined for each patient. When referring to applying overlapping forces, the magnetic stimulation system 104 of the portable NEST system 100 may use any number of magnets such as three or more magnets to apply the overlapping forces. The magnets themselves may each have their own alternating magnetic field. When the magnets are rotating near each other, the fields of the magnetic field vectors of two magnets may be added together. Thus, there may be constructive and destructive interference. Positions of magnets, magnet types, magnetic frequencies, or the phase between the magnetic fields between two magnets may be changed in order to accentuate and to stimulate some areas of the patient's brain more than other areas of the brain.

The magnetic stimulation system 104 may use various types of magnets and arrangements of magnets. The magnets of the magnetic stimulation system 104 may be arranged as or positioned in a Halbach array (e.g., arrangement of permanent magnets that may augment a magnetic field on one side of the array while cancelling field to near zero on other side of the array). The magnets may be changed around to where the magnets may accentuate the magnetic field in one direction and reduce it in another. Rotating magnets may be used along with the coil (e.g., large coil) to generate a magnetic field that has unique characteristics relating to overlapping forces approach.

The portable NEST system 100 may be used at a fixed location that may be considered generally ideal for most patients. The portable NEST system 100 may be configured to change the position of the magnetic stimulation system 104 (e.g., specifically the magnets) to treat specific regions of the patient 308 that may be based on each patient and/or a mental disorder. In general. The EEG and TMS process may be focused on the patient's brain in making treatment determinations (e.g., using the TMS treatment determination system 120). The TMS treatment determination system 120 may monitor and use neuronal activity for determination and directing the location of treatment and the treatment frequency based on the brain itself as part of the EEG and TMS process 400. Particular indications may also be used in this assessment and determination. Additionally, the magnets may be positioned to target a specific region of the patient's brain.

For example, a traumatic brain injury may be an example of a strategic location of treatment (e.g., relocation and/or movement of magnets for directing stimulation). This may be due to an actual injury to the brain itself such that stimulation of the area around the brain injury may help with the restoration of disrupted connectivity of neurons because some neurons and their networks are damaged, killed or destroyed from that trauma. The TMS treatment determination system 120 may be used to determine and direct the magnetic stimulation system 104 (e.g., magnets) to be positioned to provide stimulation to the particular damaged area (e.g., magnetic field to damaged area). Fixed ideal locations (e.g., one location or two locations such as prefrontal cortex, dorsal lateral prefrontal cortex, motor cortex, and the like as described in the disclosure) may be treated where the magnets may be fixed in positions from patient to patient.

The EEG and TMS process 400 may include recording of patient's EEG and then the EEG and TMS process 400 may indicate information about the patient based on EEG (e.g., analysis of EEG data of the patient 308). The EEG and TMS process 400 may set the frequency or phase (e.g., using the TMS treatment determination system 120).

Other Neuromodulation Technologies

The portable NEST system 100 may include a neuro-EEG synchronization therapy system having a portable unit device that may provide sTMS that may relate to treatment types and protocols (e.g., using the treatment types and protocols 122 of the portable NEST system 100 and/or the treatment types and protocols of the electrophysiology database and customized TMS treatment system 200 described in the disclosure) such that the sTMS may include magnetic stimulating forces that may provide neuromodulation that may be combined with one or more therapies. Other neuromodulation technologies may be used with the portable NEST system 100. These other neuromodulation technologies may include electrical (e.g., transcranial direct current stimulation), light stimulation therapies (e.g., photons), vibration (e.g., massage), and/or similar stimulating force that may stimulate cortical tissue. The portable NEST system 100 may be configured and/or other devices may be provided in combination with the portable NEST system 100 allowing for these other neuromodulation technologies (e.g., electrical, light) to be applied with the magnetic stimulation.

As described herein other neuromodulation technologies may include virtual reality technology. For example, a patient may view a video that may contain frequencies. A light in the view or a separate light may be set to flash at the same frequency or a harmonic of the patient's brain frequency. The portable NEST system 100 may also be redesigned to incorporate these other mechanisms such as by having headphones, goggles, vibration mechanisms, or the like that may allow for the portable NEST system 100 to impart these other neuromodulation technologies or mechanisms.

In general, the EEG and TMS process 400 may utilize these other mechanisms or therapies that may relate generally to locomotive activity (e.g., rhythmic activity) because rhythmic activity may affect the patient's brain. The EEG and TMS process 400 may be used to direct a person to perform an activity that may be a harmonic of the brain in terms of frequency such that the brain's intrinsic frequency may be accentuated. The EEG and TMS process 400 may also utilize software to provide a recommendation and/or direct a patient to run at a running pace equal to the harmonic of the patient's EEG Running at this harmonic pace may accentuate the rhythmic nature of the patient's EEG more so than if the patient ran at some other random pace or frequency. The portable NEST system 100 may include a magnetic field generator with a system that may allow for performance and/or direction of a rhythmic activity at a specific frequency related to the frequency of the patient's brain (e.g., based on EEG data) such that the patient may be in sync with the magnetic field generated and the rhythmic activity.

Rotating Magnets

The portable NEST system 100 may include a neuro-EEG synchronization therapy system having a portable unit device that may provide sTMS that may relate to treatment types and protocols (e.g., using the treatment types and protocols 122 of the portable NEST system 100 and/or the treatment types and protocols of the electrophysiology database and customized TMS treatment system 200 described in the disclosure) such that the device may include rotating magnets (e.g., as part of the magnetic stimulation system 104) that may generate an alternating magnetic field for treatment of patients. The portable NEST system 100 may include the magnetic stimulation system 104 having rotating magnets to generate an alternating magnetic field that may provide the synchronized transcranial magnetic stimulation based on the determined TMS treatment. As described, the magnetic stimulation system 104 of the portable NEST system 100 may include rotating magnets to be used for general wellness. The magnetic stimulation system 104 may use rotating permanent magnets rather than pulses from a large magnetic coil for producing low-level magnetic stimulation. The magnetic stimulation system 104 may include and use low energy rotating or moving permanent magnets that may generate an alternating magnetic field. The magnetic stimulation system 104 may use the rotating magnets to generate rTMS for treating the brain. The magnetic stimulation system 104 may utilize rotating magnets to stimulate the brain for a number of potential purposes, including the treatment of various mental disorders. Further, the magnetic stimulation system 104 may use rotating magnets to provide alternating magnetic fields that may treat the brain and may be used to treat other medical issues such as improve blood flow (e.g., blood flow in the skin). The magnetic field generated by the magnets may also be used for treating soreness and other ailments.

The magnetic stimulation system 104 may use one or more magnets (e.g., rotating cylindrical permanent magnets) that may be diametrically magnetized (e.g., neodymium The magnets may be disc magnets such as pie-shaped types of magnets which may move North, South, North, South, North, South to rotate the disc magnets. The disc magnets may provide similar results to other magnets but may not be quite as powerful. Other magnet approaches, as described in the disclosure, may include an arrangement of the magnets as a Halbach array that may allow for increased magnetic field on one side of the array and reduced magnetic field on the other side of the array (e.g., cancelled magnetic field to near zero on the other side of the array). The magnets may rotate and/or move in any variety of directions to generate the magnetic field such as moving the magnets radially, in and out, across, rotating, etc. In some examples, the magnetic stimulation system 104 may maintain one or more of the same diametrically magnetized cylindrical magnets, which may be an efficient way to generate a sinusoidal magnetic field.

As shown in FIG. 2, the magnetic stimulation system 104 (e.g., magnets) may be directed by the TMS treatment determination system 120 (e.g., software-related systems) and the NEST controller 118, and the magnetic stimulation system 104 may be indirectly managed/directed by the electrophysiology database and customized TMS treatment system 200 in terms of treatment parameters as part of the EEG and TMS process 400. These treatment parameters may include the location of application of magnetic stimulation, the frequency of magnetic stimulation, the strength of magnetic stimulation, and the like which may relate to the control of the magnets in terms of positioning, movement direction, speed, etc.

Safety Features to Prevent Misuse

The portable NEST system 100 may have the safety feature system 100 that includes safety features for preventing misuse of a TMS therapy (e.g., preventing misuse of the portable NEST system 100). In general, the safety feature system 110 may prevent misuse of TMS with respect to safety limitations. TMS-type devices may require strict and effective safety features to prevent misuse (intentional or otherwise). In some examples, trials and studies may have been about thirty minute sessions with stimulation for about sixty seconds or every minute. One of the safety limitations of TMS may be that each patient may only stimulate for about five to six seconds per minute where there may be thirty cycles for the thirty-minute session. This is to prevent overheating of brain tissue with extended stimulation, which may cause some significant damage if stimulation is applied for too long. The safety feature system 110 of the portable NEST system 100 may limit stimulation to relatively low level of energy (e.g., less than about one percent of what a typical TMS device provides). This safety feature may prevent overheating of the brain tissue such that the device could be used for hours, days, or indefinitely.

The portable NEST system 100 may allow for treatment about once a day. The safety feature system 110 may provide the ability to restrict treatment to be only allowed for thirty minutes per day and each patient user may have their own unique ID. The safety feature system 110 may have a phone or mobile application that may not allow patients to use the portable NEST system 100 more than once per day (e.g., user ID may be restricted for the user ID associated with patient on the phone application). The magnets themselves may have a natural maximum energy stimulation that they can provide such that over stimulation may be avoided. In addition, the stimulation from the magnets may be weak enough such that the patient could use the portable NEST system 100 (even without the safety restrictions) for longer periods of time without any safety risk. The safety feature system 110 may provide a TMS-related work threshold test where the portable NEST system 100 may be positioned over the patient's motor cortex and pulses may be generated looking for whether the patient's thumb twitches. Then, the safety feature system 110 and the TMS treatment determination system 120 may be used to adjust the amplitude based on the results of the TMS-related work threshold test. This may be an example of the energy level that may be set for the patient (e.g., with respect to the patient user ID) when using the portable NEST system 100. In general, the portable NEST system 100 may be considered safer than most conventional TMS devices in that the portable NEST system 100 may avoid dealing with conventional TMS device-type problems such as overcharging of a capacitor causing the capacitor to explode or fail, too much current being sent, and overheating coil that may cause the coil to melt or fail to function as required. Further, the portable NEST system 100 may use the safety feature system 110 to provide additional safety precautions as described in the disclosure.

The portable NEST system 100 (e.g., in the form of a portable unit device) may include the safety feature system 110 having safety features that may prevent misuse of TMS therapy for the device. There are various examples of these safety features as described in this disclosure. Another example safety feature may be tamper proofing (e.g., tamper resistance) for the portable NEST system 100.

Manufacturing by 3D Printer

The portable NEST system 100 may be manufactured by using 3-dimensional (3D) printing. The portable NEST system 100 may be produced by 3d printing to create a rotating NEST magnet headset device. A 3D printing manufacturing system may include capabilities of producing the portable NEST system 100 as being inexpensively printed on a 3D printer while being interfaced to a centralized and highly adaptable control system. The 3D printing system may have a control system allowing for accurate calibration of the 3D printed portable NEST system and the production of new parts on-demand to make the portable NEST system of sufficient quality to allow for low frequency, low-intensity TMS therapy for a prolonged period of time. In addition, the 3D printing system may include calibration components that may include artificial intelligence-based systems such as neural networks, hybrid neural networks, and advanced regression analysis. These systems may avoid variable quality of the produced or manufactured portable NEST system 100 providing a consistent level of clinical treatment. The 3D printing manufacturing system may produce the portable NEST system 100 having material with flexibility as well as including mechanisms (e.g., springs) for adjusting the fit of the portable NEST system 100 with patients having varying head sizes. For example, a spring may be used with the magnets providing flexibility in moving the magnets in and out with respect to the patient's head. The 3D printing system may be used to create the portable NEST system 100 as having a tight fit for patients such that the 3D printing may be customized for creating devices for each patient (e.g., based on measurements of patient's head). For example, a 3D laser scanner may be used to measure the dimensions, size, or shape of a person's head, and the 3D printer may be programmed to create the portable NEST system (e.g., portable NEST system 100) so that the portable NEST system may fit the head of the person snugly, allowing the magnets to be as close as possible to the person's scalp, and ensuring the portable NEST system is as comfortable and light as possible.

The portable NEST system 100 (e.g., portable unit device) may be manufactured by using 3D printing. The portable unit device as a rotating NEST magnet headset may be produced by a 3D printer.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein might be employed in practicing the invention. It is intended that the claims define the scope of the invention and that methods and structures within the scope of these claims and their equivalents be covered thereby.

1. A transcranial magnetic stimulation (TMS) device comprising:
a neuro-electroencephalogram synchronization therapy (NEST) headset operable to capture electroencephalogram (EEG) data;
an electrophysiology database and customized TMS treatment system, in communication with the NEST headset, wherein the electrophysiology database and customized TMS treatment system is operable to:
receive the EEG data,
analyze the EEG data to compute analyzed EEG data, and
determine a treatment based on the analyzed EEG data; and
wherein the NEST headset comprises a plurality of magnets operable to deliver synchronized transcranial magnetic stimulation based on the treatment.

2. The device of claim 1 wherein the NEST headset is portable.

3. The device of claim 1 wherein the NEST headset further comprises a portable power source.

4. The device of claim 1 wherein the NEST headset further comprises a safety feature operable to prevent misuse.

5. The device of claim 1 wherein the plurality of magnets are a plurality of rotating magnets operable to generate an alternating magnetic field to facilitate the synchronized transcranial magnetic stimulation based on the treatment.

6. (canceled)

7. The device of claim 1 wherein the NEST headset further comprises one or more of a removable strap or a removable headband.

8. The device of claim 1 further comprising a NEST controller.

9. (canceled)

10. (canceled)

11. (canceled)

12. The device of claim 1 further comprising a base station.

13. The device of claim 12 wherein the base station has an upper surface diameter and a lower surface diameter different from the upper surface diameter, and a columnar upper surface operable to hold the headset.

14. The device of claim 13 wherein the base station further comprises a Bluetooth activator accessible from an exterior surface.

15. (canceled)

16. (canceled)

17. The device of claim 1 wherein the treatment comprises the synchronized transcranial magnetic stimulation delivered using one or more of: one or more pulse frequency values, one or more physical locations, or one or more strengths

18. The device of claim 1 wherein the treatment further comprises an additional neuromodulation stimulus comprising one or more of transcranial electrical stimulation, light stimulation, or vibrational stimulation.

19. A base station for transcranial magnetic stimulation (TMS) comprising:
a housing comprising an upper portion and a lower portion, wherein the upper portion has a columnar upper surface operable to hold a neuro-electroencephalogram synchronization therapy (NEST) headset; and
a therapy controller operable to control a therapy session of the NEST headset.

20. The base station of claim 19 further comprising a Bluetooth activator accessible from an exterior surface.

21. The base station of claim 19 further comprising an energy source operable to couple with a battery of the NEST headset to recharge the battery

22. The base station of claim 19 wherein the base station is portable.

23. The base station of claim 19 further comprising a cable operable to connect to the NEST headset.

24. The base station of claim 19 wherein a first diameter across a bottom surface of the housing is wider than a second diameter across the upper surface.

25. A method for manufacturing a neuro-electroencephalogram synchronization therapy (NEST) headset comprising:
calibrating a manufacturing quality of the NEST headset using one or more of a neural network, a hybrid neural network, or advanced regression analysis; and
producing the NEST headset using 3D printing based on the manufacturing quality.

26. The method of claim 25 further comprising:
measuring dimensions of a head of a person using a 3D laser scanner; and
producing the NEST headset based on the dimensions of the head of the person.
